(19) United States
(12) Patent Application Publication
(10) Pub. No.: US 2024/0207199 A1
(43) Pub. Date: Jun. 27, 2024

(54) FLUOROCARBON COMPOSITIONS AND METHODS FOR ENHANCING IMMUNOTHERAPY

(71) Applicant: NuvOx Pharma LLC, Tucson, AZ (US)

(72) Inventor: Evan C. Unger, Tucson, AZ (US)

(21) Appl. No.: 18/287,301

(22) PCT Filed: Apr. 29, 2022
(86) PCT No.: PCT/US22/27140

§ 371 Date: Oct. 18, 2023

Related U.S. Application Data
(60) Provisional application No. 63/182,030, filed on Apr. 30, 2021.

(51) Int. Cl. A61K 31/02; A61K 9/107; A61K 39/00; A61K 45/06; A61P 35/00; C07K 16/28

(52) U.S. Cl. CPC A61K 31/02; A61K 9/107; A61K 45/06; A61P 35/00; C07K 16/2818; C07K 16/2827; A61K 2039/505

(57) ABSTRACT

The invention provides pharmaceutical compositions of fluorocarbon emulsions and methods thereof that are useful for treating cancer, in particular in enhancing the effect of immunotherapy in cancer treatment.

### PRIORITY CLAIMS AND RELATED PATENT APPLICATIONS

This application claims the benefit of priority to U.S. Provisional Application Ser. No. 63/182,030, filed on Apr. 30, 2021, the entire content of which is incorporated herein by reference.

### TECHNICAL FIELDS OF THE INVENTION

This invention relates to pharmaceutical compositions and methods of their preparation and therapeutic use. More particularly, the invention relates to pharmaceutical compositions of fluorocarbon emulsions and methods for treating cancers, in particular in enhancing the effect of immunotherapy in cancer treatment.

### BACKGROUND OF THE INVENTION

Cancer immunotherapy, a.k.a. immuno-oncology, is a form of cancer treatments that elicit the body's own immune system to treat cancer. Immunotherapy has been approved by the FDA and other regulatory authorities for treating several types of cancer. Cancer immunotherapy comes in a variety of forms and may be given alone or in combination with other cancer treatments. Current forms of immunotherapy include targeted antibodies, cancer vaccines, adoptive cell transfer, tumor-infecting viruses, checkpoint inhibitors, cytokines, and adjuvants. Many immunotherapy treatments for preventing, managing, or treating different cancers can also be used in combination with surgery, chemotherapy, radiation, or targeted therapies to improve treatment outcome.

Targeted antibodies are proteins produced by the immune system that can be customized to target specific markers (i.e., antigens) on cancer cells, in order to disrupt cancerous activity, especially unrestrained growth. Some targeted antibody-based immunotherapies are typically based on monoclonal antibodies.

Immunomodulators, such as checkpoint inhibitors, cytokines and interferons, target the molecules on either immune or cancer cells that tell immune cells when to start or stop attacking a cancer cell. Cytokines are messenger molecules that regulate cell maturation, growth, and responsiveness. Interferons (IFN) are a type of cytokine that disrupt the division of cancer cells and slow tumor growth. Interleukins (IL) are cytokines that help immune cells grow and divide more quickly. Adjuvants are immune system agents that can stimulate pathways to provide longer protection or produce more antibodies (they are often used in vaccines, but may also be used alone).

Examples of immunotherapies include: monoclonal antibody therapies such as rituximab (RITUXAN), alemtuzumab (CAMPATH) and ipilimumab (Yervoy®); non-specific immunotherapies and adjuvants include BCG, interleukin-2 (IL-2), and interferon-alfa; immunomodulating drugs, for instance, thalidomide and lenalidomide (REVLIMID); and cancer vaccines such as PROVENGE vaccine for advanced prostate cancer. Other immune modulating drugs include inhibitors of PD-L1 expression.

Despite recent advances in immunotherapy, there remains an urgent need for technologies that can achieve more effective immunotherapy against cancers.

### SUMMARY OF THE INVENTION

The invention is based in part on the unexpected discovery that pharmaceutical compositions of certain fluorocarbons with boiling points between about −36° C. to about 100° C., for example, dodecafluoropentane emulsion (DDFPe), can dramatically and beneficially augment the effects of immunotherapy against cancers. In particular, as disclosed herein, administration of such fluorocarbons increases the activity of the immunotherapy as the immune cells are more active in an oxidative environment achieved through tumor reoxygenation as effected by the fluorocarbon.

In one aspect, the invention generally relates to a method for augmenting effects of immunotherapy or reducing resistance to immunotherapy in cancer. The method comprises administration to a subject in need thereof a pharmaceutical composition comprising a fluorocarbon having a boiling point between about −36° C. to about 100° C., and a pharmaceutically acceptable carrier or excipient. In certain embodiments, the boiling point of the fluorocarbon used is preferably between about −4° C. and about 60° C. In certain embodiments, the boiling point of the fluorocarbon used is preferably between about 28° C. and about 60° C.

In another aspect, the invention generally relates to a method for treating cancer. The method comprises administrating to a subject in need thereof an immunotherapy concurrently with a pharmaceutical composition comprising a fluorocarbon having a boiling point between about −36° C. to about 100° C., and a pharmaceutically acceptable carrier or excipient.

In yet another aspect, the invention generally relates to a unit dosage form of a pharmaceutical composition in the form of an emulsion comprising a dosage of a fluorocarbon having a boiling point between about −36° C. and about +100° C. therapeutically effective to treat cancer, or a related disease or disorder thereof, in a mammal, including a human, and a pharmaceutically acceptable carrier or excipient.

### BRIEF DESCRIPTION OF THE DRAWINGS

FIG. 1 shows exemplary date on the effects of NanO₂+PD-L1 on tumor burden, where NanO₂+PD-L1 resulted in significantly slower tumor growth than PD-L1 alone. ** p<0.01.

FIG. 2 shows exemplary date on the effects of NanO₂+PD-L1 on tumor burden, where the results demonstrated significantly smaller metastatic burden than PD-L1 alone. *p<0.05.

### DEFINITIONS

Unless defined otherwise, all technical and scientific terms used herein have the same meaning as commonly understood by one of ordinary skill in the art to which this invention belongs.

As used herein, the term “emulsion” refers to a suspension or emulsion of nanodroplets or microbubbles, including phase-shift microbubbles (PSMB), in aqueous media. Nanodroplets refers to submicron droplets comprising a liquid fluorocarbon, e.g., ranging from 4 carbon to 8 carbons in length.

As used herein, “administration” of a disclosed compound or composition encompasses the delivery to a subject of a pharmaceutical composition using any suitable formulation or route of administration, as discussed herein.

As used herein, the terms “effective amount” or “therapeutically effective amount” refer to that amount of a compound or pharmaceutical composition described herein that is sufficient to effect the intended benefit including, but not limited to, disease treatment, as illustrated herein. The therapeutically effective amount can vary depending upon the intended application, or the subject and disease condition being treated, e.g., the desired biological endpoint, the pharmacokinetics of the compound, the disease being treated, the mode of administration, and the weight and age of the patient, which can readily be determined by one of ordinary skill in the art. The specific dose will vary depending on, for example, the particular compounds chosen, the species of subject and their age/existing health conditions or risk for health conditions, the dosing regimen to be followed, the severity of the disease, whether it is administered in combination with other agents, timing of administration, the tissue to which it is administered or targeted, and the physical delivery system in which it is carried.

As used herein, the terms “treatment” or “treating” a disease or disorder refers to a method of reducing, delaying or ameliorating such a condition before or after it has occurred. Treatment may be directed at one or more effects or symptoms of a disease and/or the underlying pathology. For instance, treatment herein may achieve an increase in a subject's oxygen saturation level or an improvement or restoration of oxygen supply. Treatment is aimed to obtain beneficial or desired results including, but not limited to, therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder. For prophylactic benefit, the pharmaceutical compounds and/or compositions can be administered to a patient at risk of developing a particular disease or condition, or to a patient reporting one or more of the physiological symptoms of a disease or condition, even though a diagnosis of this disease or condition may not have been made. The treatment can be any reduction and can be, but is not limited to, the complete ablation of the disease or condition or the symptoms of the disease or condition. As compared with an equivalent untreated control, such reduction or degree of prevention is at least 5%, 10%, 20%, 40%, 50%, 60%, 80%, 90%, 95%, or 100% as measured by any standard technique.

As used herein, the term “therapeutic effect” refers to a therapeutic benefit and/or a prophylactic benefit as described herein. A prophylactic effect includes delaying or eliminating the appearance of a disease or condition, delaying or eliminating the onset of symptoms of a disease or condition, slowing, halting, or reversing the progression of a disease or condition, or any combination thereof.

As used herein, the term “pharmaceutically acceptable” excipient, carrier, or diluent refers to a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not unacceptably injurious to the patient.

As used herein, the term “subject” refers to any animal (e.g., a mammal), including, but not limited to humans, non-human primates, rodents, and the like, including both food and companion animals, which is to be the recipient of a particular treatment. Typically, the terms “subject” and “patient” are used interchangeably herein in reference to a human or veterinary subject.

As used herein, the “low dosage” refers to at least 5% less (e.g., at least 10%, 20%, 50%, 80%, 90%, or even 95%) than the lowest standard recommended dosage of a particular compound formulated for a given route of administration for treatment of any human disease or condition. For example, a low dosage of an agent that reduces glucose levels and that is formulated for administration by inhalation will differ from a low dosage of the same agent formulated for oral administration.

As used herein, the “high dosage” is meant at least 5% (e.g., at least 10%, 20%, 50%, 100%, 200%, or even 300%) more than the highest standard recommended dosage of a particular compound for treatment of any human disease or condition.

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure.

### DETAILED DESCRIPTION OF THE INVENTION

The invention relates to a surprising discovery that an oxygen therapeutic comprising a fluorocarbon emulsion can be administered to reverse or ameliorate tumor hypoxia and augment immunotherapy for cancer treatment. The invention provides compositions of certain fluorocarbons with boiling points between about −36° C. to about 100° C., for example dodecafluoropentane emulsion (DDFPe), that are useful for augmenting the effects of various types of immunotherapy for cancer treatment.

Solid tumors have aberrant blood supplies resulting in areas of tumor tissue with decreased perfusion and hypoxia. Hypoxia upregulates expression of genes causing an aggressive phenotype. Hypoxic tumors use anaerobic glycolysis and secrete adenosine into the extracellular matrix of the tumor tissue. Adenosine is a potent suppressor of killer T-cell lymphocytes. Killer T-cells also need oxygen in order to kill tumor cells and low oxygen concentrations within hypoxic tumors render T-cells ineffective in fighting tumors. Hypoxic tumors often develop a fibrotic extracellular matrix which makes it difficult for T-cells to infiltrate tumor tissue and pose a barrier to immunotherapy treatment of cancer.

Without wishing to be bound by the theory, administration of the fluorocarbon compositions of the invention leads to the immune cells becoming more active in an oxidative environment achieved through tumor reoxygenation, thereby increasing the activity of the immunotherapy. Administration of the fluorocarbon emulsion can be used to increase the oxygen in the tumor tissue so that immune mechanisms are accelerated and making the immune system more efficient as well as changing gene expression. By decreasing expression of hypoxia related genes, the oxygen therapeutic converts the aggressive hypoxia mediated phenotype to a less aggressive phenotype that is more easily defeated by the immune system.

Other immune modulating drugs that can be used along with the fluorocarbon emulsions include inhibitors of PD-LI expression. Monoclonal antibodies are particularly suitable for use with the fluorocarbon emulsions of the invention. The antibodies can be used as vaccines to trigger an immune response to reject the cancer. Antibodies useful in this invention include Alemtuzumab, Bevacizumab. Brentuximub vedotm, Cetuximab, Gemtuzumab ozogamicin, Ibritumomab tiuxetan, Ipilimumab, Nivolumab, Ofatumumab, Panitumumab, Rituximab, Tositumomab and Trastuzumab.

Nonspecific stimulators of the immune system can also be used with the fluorocarbon emulsions of the invention. Examples include cytokines (e.g., interleukins), interferons (e.g., interferon-alpha) and thymic peptides (e.g. thymosin alpha-1).

The invention can also be used with adoptive T-cell therapy anti-CD47 antibodies, AntiGD2 antibodies, immune checkpoint blockade and EGF receptor antibodies.

The invention is useful for treatment of a wide variety of cancers including but not limited to small cell lung cancer, melanoma, non-small cell cancer, head and neck squamous cell cancer, classic Hodgkin lymphoma, primary mediastinal large B cell lymphoma, urothelial carcinoma, microsatellite instability-high mismatch repair deficient cancer, solid tumors that have progressed following prior treatments which have no satisfactory treatment options, colorectal cancer, gastric cancer, esophageal cancer, cervical cancer, hepatocellular carcinoma, Merkel cell carcinoma, renal cell carcinoma, endometrial carcinoma, tumor mutational burden-high cancer, cutaneous squamous cell carcinoma, breast carcinoma including triple-negative breast cancer, pancreatic cancer, malignant pleural mesothelioma, glioblastoma and soft tissue sarcoma.

In general, to practice the invention and derive the benefits therefrom the oxygen therapeutic comprising a fluorocarbon is administered intravenously either via infusion or bolus in association with the use of an immunotherapy drug to treat cancer.

Checkpoint proteins, such as PD-L1 on tumor cells and PD-1 on T cells are important factors regulating immune responses. When PD-1 on T cells binds to PD-L1 on tumor cells this keeps T cells from killing the tumor cells. In the setting of hypoxia PD-L1 is overexpressed on monocytes and PD-1 is overexpressed on CD8⁺ T cells (Cubillos-Zapata C, Hypoxia-induced PD-L1/PD-1 crosstalk impairs T-cell function in sleep apnea. Eur Respir J 2017; 50: 1700833 [https://doi.org/10.1183/13993003.00833-2017]).

Immune checkpoint inhibitors allow the T cells to kill tumor cells (https://www.cancer.gov/about-cancer/treatment/types/immunotherapy/checkpoint-inhibitors). Hypoxia, however, diminishes the therapeutic effects of checkpoint inhibitors through multiple mechanisms. Immunotherapy drugs include check point inhibitors which block checkpoints enabling T-cells to be more active in fighting cancer. PD-1 inhibitors include KEYTRUDA (pembrolizumab), OPDIVO (nivolumab) and Libtayo (cemiplimab). PD-L1 inhibitors include Tecentriq (atezolizumab) Bavencio (avelumab) and Imfinzi (durvalumab). CTLA-4 inhibitors include Yervoy (ipilimumab) and tremelimumab. All of these drugs are antibodies and are administered intravenously.

In one aspect, the invention generally relates to a method for augmenting effects of immunotherapy or reducing resistance to immunotherapy in cancer. The method comprises administration to a subject in need thereof a pharmaceutical composition comprising a fluorocarbon having a boiling point between about −36° C. to about 100° C., and a pharmaceutically acceptable carrier or excipient. In certain embodiments, the boiling point of the fluorocarbon used is preferably between about −4° C. and about 60° C. In certain embodiments, the boiling point of the fluorocarbon used is preferably between about 28° C.and about 60°.

In another aspect, the invention generally relates to a method for treating cancer. The method comprises administrating to a subject in need thereof an immunotherapy concurrently with a pharmaceutical composition comprising a fluorocarbon having a boiling point between about −36° C. to about 100° C., and a pharmaceutically acceptable carrier or excipient.

In certain embodiments, the fluorocarbon has from about 4 to about 8 (e.g., 4, 5, 6, 7 or 8) carbons in length with from about 10 to about 18 fluorine atoms.

In certain embodiments, the pharmaceutical composition is an emulsion.

In certain embodiments, the immunotherapy comprises a targeted antibody therapy.

In certain embodiments, the immunotherapy comprises monoclonal antibody therapy. In certain embodiments, the monoclonal antibody is a checkpoint inhibitor to PD-L1, CTLA-4 or PD-1.

In certain embodiments, the immunotherapy comprises a non-specific immunotherapy.

In certain embodiments, the immunotherapy comprises immunomodulators selected from checkpoint inhibitors, cytokines, thymic peptides and interferons.

In certain embodiments, the immunotherapy comprises adoptive T-cell therapy.

In certain embodiments, the cancer being treated is a solid tumor.

In certain embodiments, the cancer being treated is a liquid tumor.

In certain embodiments, the cancer is selected from: small cell lung cancer, melanoma, non-small cell cancer, head and neck squamous cell cancer, classic Hodgkin lymphoma, primary mediastinal large B cell lymphoma, urothelial carcinoma, microsatellite instability-high mismatch repair deficient cancer, colorectal cancer, gastric cancer, esophageal cancer, cervical cancer, hepatocellular carcinoma, Merkel cell carcinoma, renal cell carcinoma, endometrial carcinoma, tumor mutational burden-high cancer, cutaneous squamous cell carcinoma, breast carcinoma including triple-negative breast cancer, pancreatic cancer, malignant pleural mesothelioma, glioblastoma and soft tissue sarcoma.

In certain embodiments, the fluorocarbon is provided as nanodroplets or in microbubbles. In certain embodiments, the emulsion comprises nanodroplets or microbubbles less than 1 micron in size. In certain embodiments, the emulsion comprises nanodroplets or microbubbles greater than 1 micron in size. In certain embodiments, the nanodroplets or in microbubbles have size from about 0.5 microns to about 5 microns.

In certain embodiments, the fluorocarbon comprises perfluoropropane, perfluorobutane, perfluoropentane, perfluorohexane, perfluoroheptane, perfluorooctane, or a mixture of two of more thereof. In certain embodiments, the pharmaceutical composition utilizes perfluoropentane. Perfluoropentane may comprise isomers of dodecafluoro-n-pentane (dodecafluoropentane) and dodecafluoro-iso-pentane. In certain embodiments, the fluorocarbon is dodecafluoropentane.

In certain embodiments, the emulsion has from about 0.5 to about 20% w/vol of fluorocarbon. In certain embodiments, the emulsion has between about 1 and about 10% w/vol fluorocarbon. In certain embodiments, the emulsion has between about 1 and about 5% w/vol fluorocarbon. In certain embodiments, the emulsion has between about 3 and about 7% w/vol fluorocarbon. In certain embodiments, the emulsion has from about 0.5 to about 2% w/vol of fluorocarbon. In certain embodiments, the emulsion has between about 1 and about 3% w/vol fluorocarbon. In certain embodiments, the emulsion has between about 2 and about 4% w/vol fluorocarbon. In certain embodiments, the emulsion has between about 3 and about 5% w/vol fluorocarbon.

In certain embodiments, the pharmaceutical composition comprises one or more phospholipids having carbon chains ranging from about 12 carbons to about 18 carbons in length.

In certain embodiments, the phospholipids accounts for a weight percent in the pharmaceutical composition from about 0.10% to about 7.5% (e.g., from about 0.10% to about 1.0%, from about 1.0% to about 2.5%, from about 2.5% to about 5.0%).

In certain embodiments, the fluorocarbon is stabilized by one or more surfactants. For example, surfactants may be one or more fluorosurfactants such as PEG-Telomer-B, CAPSTONE, diacylglycerophospholipids, cholesterol, and/or other surfactants known in the art. In certain embodiments, the surfactant(s) utilized comprise one or more fluorosurfactants and one or more phospholipids.

In certain embodiments, the fluorocarbon is stabilized by perfluorocarbon-oligoethoxyalcohol, such as PEG-Telomer-B or Capstone, without a phospholipid. In certain embodiments, the fluorocarbon is stabilized by PEG-Telomer-B and a phospholipid. In certain embodiments, the fluorocarbon is stabilized by a phospholipid.

In certain embodiments, the surfactant(s) is incorporated into the emulsion in amounts ranging from about 0.1% weight volume to about 10% weight volume (e.g., about 0.1% weight volume to about 1.0% weight volume, about 1.0% weight volume to about 2.0% weight volume, about 2.0% weight volume to about 5.0% weight volume). In certain embodiments, the surfactant(s) is incorporated into the emulsion in amounts ranging from about 0.2% w/vol to about 2% w/vol.

In certain embodiments, the pharmaceutical composition is administered intravenously. In certain embodiments, the pharmaceutical composition may be administered as an IV bolus. In certain embodiments, the pharmaceutical composition may be administered by sustained IV infusion. In certain embodiments, the pharmaceutical composition is injected intravenously via bolus or slow IV push over about 3 to about 5 minutes at doses ranging from about 0.2 mg/kg to about 20 mg/kg (e.g., about 0.2 mg/kg to about 10 mg/kg, about 0.2 mg/kg to about 5 mg/kg, about 0.2 mg/kg to about 2 mg/kg, about 1 mg/kg to about 20 mg/kg, about 5 mg/kg to about 20 mg/kg, about 10 mg/kg to about 20 mg/kg).

In certain embodiments, the pharmaceutical composition is injected intravenously as a sustained IV infusion at a rate of from about 0.5 mg/kg/hour up to about 7.0 mg/kg/hour (e.g., about 0.5 mg/kg/hour up to about 5.0 mg/kg/hour, about 0.5 mg/kg/hour up to about 3.05 mg/kg/hour, about 0.5 mg/kg/hour up to about 2.0 mg/kg/hour, about 1 mg/kg/hour up to about 7.0 mg/kg/hour, about 2 mg/kg/hour up to about 7.0 mg/kg/hour, about 3 mg/kg/hour up to about 7.0 mg/kg/hour).

The concentration of fluorocarbon in the emulsion can be increased, for example, up to about 60% weight/vol if desired, to minimize the volume injected.

In certain embodiments, the pharmaceutical composition is administered as an IV infusion at a rate of from about 2.0 mg/kg to about 40 mg/kg per hour (e.g., about 2.0 mg/kg to about 20 mg/kg per hour, about 2.0 mg/kg to about 10 mg/kg per hour, about 2.0 mg/kg to about 5 mg/kg per hour, about 2.0 mg/kg to about 2 mg/kg per hour, about 1 mg/kg to about 40 mg/kg per hour, about 5 mg/kg to about 40 mg/kg per hour, about 10 mg/kg to about 40 mg/kg per hour, about 1 mg/kg to about 10 mg/kg per hour, about 5 mg/kg to about 20 mg/kg per hour).

In certain embodiments, a dose of the pharmaceutical composition is repeated as needed, for example from 1 to about 50 times (e.g., about from 1 to about 25 times, from 1 to about 10 times, from 1 to about 5 times, from 1 to about 3 times, from 2 to about 10 times).

In certain embodiments, the method comprises administering the subject a third therapeutic agent, before, during or after the administration of the pharmaceutical composition of fluorocarbon and/or immunotherapy, wherein the third therapeutic agent is a chemotherapeutic agent.

In yet another aspect, the invention generally relates to a unit dosage form of a pharmaceutical composition in the form of an emulsion comprising a dosage of a fluorocarbon having a boiling point between about −36° C. and about +100° C. therapeutically effective to treat cancer, or a related disease or disorder thereof, in a mammal, including a human, and a pharmaceutically acceptable carrier or excipient. The unit dosage form may be presented as a single dosage or in a multidosage format.

As disclosed herein, fluorocarbons useful in the invention include perfluoropropane, perfluorobutane, perfluoropentane, perfluorohexane, perfluoroheptane and perfluorooctane, or a mixture of two of more thereof. In certain embodiments, the pharmaceutical composition utilizes perfluorohexane and/or perfluoropentane. In certain embodiments, the pharmaceutical composition utilizes perfluoropentane. Perfluoropentane may comprise isomers of dodecafluoro-n-pentane (dodecafluoropentane) and dodecafluoro-iso-pentane. In certain embodiments, the fluorocarbon is dodecafluoropentane. In certain embodiments of the unit dosage form, the fluorocarbon is dodecafluoropentane.

Any suitable therapeutically effective unit dosage form may be employed, for example, comprising about 1% to about 10% w/vol of the fluorocarbon. In certain embodiments, the unit dosage form comprises about 2% to about 7% w/vol of the fluorocarbon. In certain embodiments, the unit dosage form comprises from about 1 mg/kg body weight to about 7 mg/kg (e.g., about 1 mg/kg body weight to about 5 mg/kg, about 1 mg/kg body weight to about 3 mg/kg, about 2 mg/kg body weight to about 7 mg/kg, about 3 mg/kg body weight to about 7 mg/kg) body weight of fluorocarbon.

The following examples are presented to further illustrate to persons skilled in the art how to make and use the invention. These examples are not intended as a limitation, however, upon the scope of the invention.

### EXAMPLES

### Example 1. Anti-PD-L1 and NanO₂ in Immuno-Oncology

A study was performed in Balb/cJ mice with triple negative breast cancer (TNBC) (Jackson Laboratories, Bar Harbor, Maine). Mice were implanted with 5×10⁴ cells of 4T1 TNBC tumor cells. When the tumors were palpable (50 mm size) the animals were treated. There were five animals treated with placebo, 10 treated with PD-L1+placebo and 10 treated with PD-L1+NanO₂ (2% w/vol dodecafluoropentane emulsion) at a dose of 0.6 mL/kg. The PD-L1 was BioXcell #BE0101 the antibody buffer diluent used in the experiment was BioXcell #IP0070 (BioXcell, Lebanon, New Hampshire).

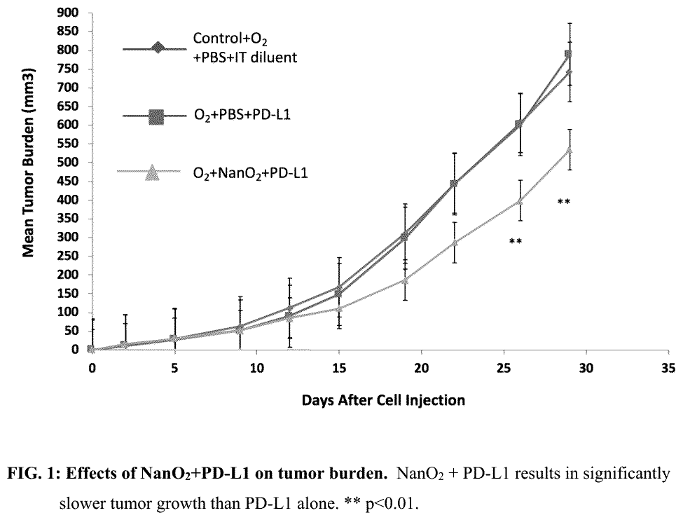

Control and PD-L1 comparison groups were administered normal saline as placebo at dose=1 mg/mL, retro-orbitally. All animals were placed in a chamber and breathed 100% normobaric oxygen for 60 minutes. The animals were removed from the chamber and injected with the relevant test articles. All animals received treatments (oxygen breathing+injection of test articles) on Monday, Wednesday and Friday of the first week (three treatments total). Tumor sizes were measured bi-dimensionally for thirty days. Tumor burden was calculated according to the formula: V=(dₛₕₒᵣₜ)2×(d_{long})/2. As shown in FIG. 1, below, the NanO₂+PD-L1 group had a highly significantly slower increase in tumor size as compared to PD-L1 alone.

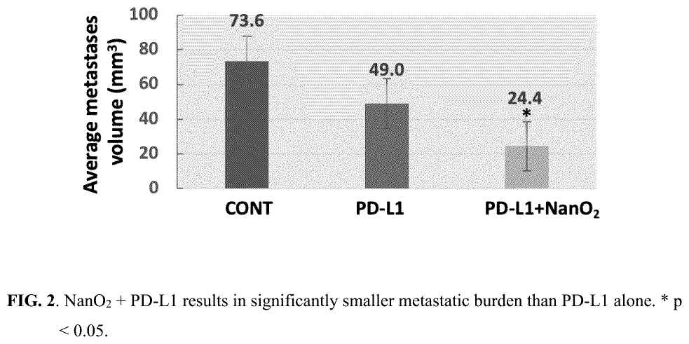

After the animals were sacrificed the volume of metastatic disease was calculated by measuring the bidimensional measurement of metastatic lesions according to the formula as above. Most metastatic events occurred in the lungs, As shown in FIG. 2, below, the NanO₂ combination with PD-L1 group had significantly smaller burden of metastatic disease. PD-L1 alone was not significantly different from control.

There was no change in the individual mouse weight in each treatment group.

In a tumor regression study where the tumors were grown up to 600 mm³ prior to treatment, animals that were treated with the combination of NanO₂+PD-L1 antibody showed a tumor volume that was 511±70 mm³ versus the tumor volumes from animals treated with the antibody alone measured 763±117 mm³ at day 28 post tumor implantation. Survival in the combination group treated with NanO₂ was 75% alive at day 35 as compared to 50% at day 35 for PD-L1 alone.

### Prophetic Example 1

A patient with melanoma is treated with 200 mg IV Keytruda every 3 weeks. Four-hours prior to administration of Keytruda the patient is administered an IV loading dose of 0.17 mL/kg of DDFPe (2% w/vol emulsion), followed by sustained IV infusion of DDFPe at a rate of 0.1 mL/kg per hour for four-hours prior to administration of Keytruda and for four-hours after administration of Keytruda while breathing oxygen with a non-rebreather oxygen mask.

### Prophetic Example 2

A patient with non-small cell lung cancer is treated with 400 mg IV Keytruda every 6 weeks. Twenty-four hours prior to receiving Keytruda the patient received an IV loading dose of 0.1 mL/kg of DDFPe (2% w/vol emulsion) and a 24-hour IV infusion of DDFPe while breathing oxygen—4 Liters/minute via nasal prong. After IV administration of Keytruda the patient receives 4-hours more of infusion of DDFPe while continuing to breath oxygen.

### Prophetic Example 3

A patient with malignant pleural mesothelioma is treated with ipilimumab 360 mg IV every 3 weeks. The patient is administered an IV bolus of DDFPe 2-hours prior to ipilimumab while breathing oxygen—4 L/min via nasal prongs and continues to breath oxygen for 2-hours after administration of ipilimumab.

### Prophetic Example 4

A patient with advanced renal cell carcinoma is administered 3 mg/kg ipilimumab followed by ipilimumab 1 mg/kg on the same day every 3 weeks for 4 doses, then 240 mg every 2 weeks. Two hours prior to each dose of ipilimumab the patient is administered a slow IV push of 0.25 mL/kg DDFPe while breathing oxygen—2 L/min via nasal prongs and continues to breath supplemental oxygen for 2-hours after each ipilimumab treatment.

### Prophetic Example 5

A patient with hepatocellular carcinoma is treated with Opdivo as a 240 mg dose every two weeks via 30-minute intravenous infusion. Ninety minutes prior to commencing the infusion of Opdivo the patient receives an IV bolus of 0.17 mL/kg of DDFPe while breathing oxygen 4L/min via nasal prong. An IV infusion of DDFPe is commenced following the bolus loading dose at a rate of 0.1 mL/kg. The infusion of DDFPe continues while the Opdivo is infused and the patient continues to breath oxygen for 30 minutes after the infusion of Opdivo has ended.

### Prophetic Example 6

A patient with gastric cancer is treated with Opdivo 240 mg every 2 weeks (30-minute intravenous infusion) with fluoropyrimidine- and platinum-containing chemotherapy every 2 weeks. Ninety minutes prior to commencing the infusion of Opdivo the patient receives an IV bolus of 0.25 mL/kg of 5% w/vol emulsion of perfluorohexane while breathing oxygen 4L/min via nasal prong. An IV infusion of perfluorohexane emulsion is commenced following the bolus loading dose at a rate of 2.0 mL/kg. The infusion of perfluorohexane emulsion continues while the Opdivo is infused and the patient continues to breath oxygen for 30 minutes after the infusion of Opdivo has ended.

### Prophetic Example 7

A patient with cutaneous squamous cell carcinoma is treated with Libatyo, 3 mg/kg every 2 weeks as an IV infusion for 110 weeks. Perfluorooctane emulsion, 10% weight/volume was administered as a slow IV push at a dose of 0.25 mL/kg 2 hours prior to each infusion of Libatyo while the patient breathed room air.

### Prophetic Example 8

A patient with advanced basal cell carcinoma is treated with Libatayo, 350 mg IV every 3 weeks, for a total of 12 doses over a period of 42 weeks. DDFPe (2% w/vol emulsion) as administered as a slow IV push at a dose of 0.25 mL/kg 2 hours prior to each infusion of Libatayo while the patient breathed oxygen via nasal prong at 2 L/min.

### Prophetic Example 9

A patient with non-squamous non-small cell lung cancer with no EGFR or ALK genomic tumor aberrations is treated with Tecentriq in combination with bevacizumab, paclitaxel, and carboplatin as first line therapy. Tecentriq is administered as an IV infusion at a dose of 840 mg every 2 weeks and is administered prior to chemotherapy and bevacizumab when administered on the same day. Four-hours prior to each dose of Tecentriq, DDFPe (2% w/vol emulsion) is administered as a 0.25 mL/kg loading dose via slow IV push and as a sustained IV infusion at a dose of 0.17 mL/kg per hour until the infusion of Tecentriq. The patient is breathing oxygen during this time via face mask at 4 L/min and until cessation of infusion of Tecentriq.

Prophetic Example 10

A patient with metastatic triple negative breast cancer is treated with Tecentriq at a dose of 840 mg every 2 weeks in combination with protein-bound paclitaxel which is administered IV at a dose of 100 mg/m² on days 1, 8 and 15 of each 28-day cycle. Tecentriq is administered prior to paclitaxel when administered on the same day. DDFPe (5% w/vol emulsion) at a dose of 0.17 mL/kg is administered as a slow IV push 2 hours prior to the infusion of Tecentriq while the patient breathes oxygen 6 L/min via face mask. The patient continues to breath oxygen until cessation of the Tecentriq infusion.

### Prophetic Example 11

A patient with Merkel cell carcinoma is treated with Bavencio, 800 mg administered as an IV infusion over 60 minutes every two weeks. The patient is premedicated with an antihistamine and with acetaminophen prior to the first 4 IV infusions of Bavencio. DDFPe (2% w/vol emulsion) is administered as a slow IV push at a dose of 0.25 mL/kg 2 hours prior to the infusion of Bavencio while the patient breathes oxygen 6 L/min via face mask. The patient continues to breath oxygen until cessation of the Bavencio infusion.

### Prophetic Example 12

A patient with small cell lung cancer is treated with Imfinizi in combination with etoposide and carboplatin. Imfinizi is administered as an IV infusion at dose of 10 mg/kg every 2 weeks. DDFPe (2% w/vol emulsion) is administered as a slow IV push at a dose of 0.17 mL/kg 2 hours prior to the infusion of Imfinizi while the patient breathes oxygen 2 L/min via nasal prongs. The patient continues to breath oxygen until cessation of the Imfinizi infusion.

### Prophetic Example 13

A patient with pancreatic ductal adenocarcinoma (PDAC) it treated with Opdivo 240 mg every 2 weeks. The patient receives treatments three times a week with DDFPe on Monday, Wednesday and Friday, a 1-hour IV infusion of DDFPe at a dose of 0.3 mL/kg (2% w/vol emulsion) while breathing oxygen 6 liters per minute for two hours. On the Friday of every second week the patient receives the infusion of Opdivo 90 minutes after the infusion of DDFPe was commenced.

### Prophetic Example 14

A patient with PDAC is treated with combination therapy with immune checkpoint inhibitors to CTLA-4 and PD-1. The checkpoint inhibitors are administered together via IV infusion every 2 weeks. Prior to each infusion of the combined checkpoint inhibitors, the patient receives a slow IV push of DDFPe at a dose of 0.17 mL/kg two hours prior to infusion of the checkpoint inhibitors and breathes oxygen at 4 L/min via nasal prong and continues to breath oxygen for two hours following cessation of infusion of the checkpoint inhibitors.

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the following description, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A method for augmenting effects of immunotherapy or reducing resistance to immunotherapy in cancer, comprising administrating to a subject in need thereof who has received, is receiving or will receive such immunotherapy a pharmaceutical composition comprising a fluorocarbon having a boiling point between about −36° C. to about 100° C., and a pharmaceutically acceptable carrier or excipient.

2. A method for treating cancer, comprising administrating to a subject in need thereof an immunotherapy concurrently with a pharmaceutical composition comprising a fluorocarbon having a boiling point between about −36° C. to about 100° C., and a pharmaceutically acceptable carrier or excipient.

3. The method of claim 2, wherein the fluorocarbon comprises perfluoropropane, perfluorobutane, perfluoropentane, perfluorohexane, perfluoroheptane, perfluorooctane, or a mixture of two of more thereof, and the pharmaceutical composition is an emulsion.

4. (canceled)

5. (canceled)

6. The method of claim 3, wherein the immunotherapy comprises monoclonal antibody therapy.

7. The method of claim 6, wherein the monoclonal antibody is a checkpoint inhibitor to PD-L1, CTLA-4 or PD-1.

8. The method of claim 6, wherein the monoclonal antibody is selected from the group consisting of Alemtuzumab, Bevacizumab. Brentuximub vedotm, Cetuximab, Gemtuzumab ozogamicin, Ibritumomab tiuxetan, Ipilimumab, Nivolumab, Ofatumumab, Panitumumab, Rituximab, Tositumomab and Trastuzumab.

9. (canceled)

10. The method of claim 3, wherein the immunotherapy comprises immunomodulators selected from checkpoint inhibitors, cytokines, thymic peptides and interferons.

11. The method of claim 3, wherein the immunotherapy comprises adoptive T-cell therapy.

12. The method of claim 3, wherein the cancer being treated is a solid tumor.

13. The method of claim 3, wherein the cancer being treated is a liquid tumor.

14. The method of claim 3, wherein the cancer is selected from: small cell lung cancer, melanoma, non-small cell cancer, head and neck squamous cell cancer, classic Hodgkin lymphoma, primary mediastinal large B cell lymphoma, urothelial carcinoma, microsatellite instability-high mismatch repair deficient cancer, colorectal cancer, gastric cancer, esophageal cancer, cervical cancer, hepatocellular carcinoma, Merkel cell carcinoma, renal cell carcinoma, endometrial carcinoma, tumor mutational burden-high cancer, cutaneous squamous cell carcinoma, breast carcinoma including triple-negative breast cancer, pancreatic cancer, malignant pleural mesothelioma, glioblastoma and soft tissue sarcoma.

15. (canceled)

16. The method of claim 3, wherein the emulsion comprises nanodroplets or microbubbles less than 1 micron in size.

17. The method of claim 3, wherein the nanodroplets or in microbubbles have size from about 0.5 microns to about 5 microns.

18. (canceled)

19. The method of claim 3, wherein the fluorocarbon comprises dodecafluoropentane.

20. The method of claim 19, wherein the fluorocarbon is present at a concentration ranging from about 0.1% w/vol to about 10% w/vol.

21. (canceled)

22. (canceled)

23. The method of claim 20, wherein the pharmaceutical composition is administered intravenously.

24. The method of claim 20, wherein the pharmaceutical composition is injected intravenously via bolus or slow IV push.

25. The method of claim 20, wherein the pharmaceutical composition is administered as an IV infusion at a rate of from about 0.2 mg/kg to about 40 mg/kg per hour.

26. The method of claim 20, comprising administering the subject a third therapeutic agent, before, during or after the administration of the pharmaceutical composition of fluorocarbon and/or immunotherapy, wherein the third therapeutic agent is a chemotherapeutic agent.

27. A unit dosage form of a pharmaceutical composition in the form of an emulsion comprising a dosage of a fluorocarbon having a boiling point between about −36° C. and about +100° C. therapeutically effective to treat cancer, or a related disease or disorder thereof, in a mammal, including a human, and a pharmaceutically acceptable carrier or excipient.

28-32. (canceled)
